(19) United States
(12) Patent Application Publication
(10) Pub. No.: US 2025/0099689 A1
(43) Pub. Date: Mar. 27, 2025

(54) LABELLING ARRANGEMENT AND METHOD FOR PRODUCING A LABELLING ARRANGEMENT FOR A SYRINGE, AND SYRINGE SYSTEM

(71) Applicant: Schreiner Group GmbH & Co. KG, Oberschleissheim (DE)

(72) Inventors: Christoph STEINER, Maisach (DE); Timo HOELZL, Pfaffenhofen (DE); Stefan KRAUSS, Muenchen (DE); Michael ZAWAR, Garching (DE); Marei KREITMAIR, Schwabhausen (DE); Tanja EHREISER-PIEHLER, Neufahrn (DE); Christian LIEBL, Westbury, NY (US); Andreas HOFENAUER, Germering (DE); Christian MECHTLER, Muenchen (DE)

(73) Assignee: Schreiner Group GmbH & Co. KG, Oberschleissheim (DE)

(21) Appl. No.: 18/573,502

(22) PCT Filed: Jun. 2, 2022
(86) PCT No.: PCT/EP2022/065011

§ 371 Date: Dec. 22, 2023

(51) Int. Cl. A61M 5/32; A61M 5/50; G09F 3/00

(52) U.S. Cl. CPC A61M 5/3216; A61M 5/5086; G09F 3/0295; A61M 2005/3217

(57) ABSTRACT

A labeling arrangement for a syringe includes a label adapted to be attached to the syringe body, and a needle trap adapted to receive and secure the syringe needle after use. A first label flap is coupled to the needle trap, and a second label flap separate from the first label flap has a predetermined severable design and is adapted to be attached to a needle protection cap surrounding the syringe needle and to form an opening detection element on the syringe. The first and second label flaps are connected to each other by the label so that, when the labeling arrangement is attached to the syringe in an operational state, the second label flap is severable when the syringe is opened by removing the needle protection cap and a second label flap section together with the needle protection cap is removable from the label and the syringe body.

The present invention relates to a labeling arrangement for attachment to a syringe. The invention further relates to a syringe system with such a labeling arrangement and a corresponding manufacturing method for such a labeling arrangement.

Conventional syringes generally have a needle guard or a needle guard cap which encloses the syringe needle and protects it from unwanted contact. Needle stick protection devices or needle traps can also be provided, which are coupled to a syringe and are intended to provide protection from the syringe needle after the syringe has been used. Such systems can provide reliable protection, but may have a complex structure and are associated with correspondingly high material costs and high manufacturing costs.

It is an objective underlying the invention to contribute to reliable protection for a syringe in a simple and cost-effective manner.

The objective is achieved by the features of the independent patent claims. Advantageous embodiments are given in the respective dependent patent claims.

According to an aspect of the invention, a labeling arrangement for attachment to a syringe comprising a syringe body, a syringe needle and a needle protection cap surrounding the syringe needle, incudes a label adapted to be attached to the syringe body of the syringe. The labeling arrangement further comprises a needle trap adapted to receive and secure the syringe needle of the syringe after use. The labeling arrangement also has a first label flap which is coupled to the needle trap. In addition, the labeling arrangement has a second label flap which is separate from the first label flap, designed to be predeterminedly separable and which is set up to be attached to the needle protection cap of the syringe and to form an opening detection element on the syringe, wherein the first and second label flaps are connected to one another by means of the label, so that, with respect to an applied state in which the labeling arrangement is attached to the syringe in accordance with operation, when the syringe is opened by removing the needle protection cap, the second label flap can be separated and a section of the second label flap together with the needle protection cap can be removed from the label and the syringe body.

By means of the described labeling arrangement, reliable protection with integrated first-opening verification for a syringe can be realized in a simple and cost-effective manner. The labeling arrangement comprises a needlestick protection with the needle trap and a first-opening indicator with the second label flap, by means of which the use of the syringe or a successful opening can be clearly visibly indicated. Thus, in addition to a needlestick protection device, which is realized by the needle trap, a premature removal of the needle protection cap and thus a loss of sterility can be clearly indicated with the separately formed second label tab.

The first label flap remains on the label and the syringe body with the needle trap attached to it in order to hold and secure the syringe needle after the syringe has been used, thus preventing unwanted contact and injuries caused by the used syringe needle. Accordingly, when the syringe is opened by removing the needle protection cap, the second label flap can be severed and a section of the second label flap together with the needle protection cap can also be removed from the label and the first label flap.

The needle trap forms a needlestick protection device and is realized, for example, as a molded plastic part and connected, in particular glued, to the first label flap. The needle trap and/or the first label flap are connected to the label in an articulated manner and are approximately parallel to the needle protection cap in relation to an applied state in which the labeling arrangement is attached to the syringe during operation. When using the syringe, a user, for example a doctor or nurse, tilts the needle trap away from the needle protection cap so that the needle trap protrudes from the syringe and the needle protection cap is easily accessible. Due to the predetermined articulated connection, the needle trap protrudes at a defined angle from the needle protection cap, which can then be removed by the user.

The needle trap comprises, for example, an elongated recess for the syringe needle and one or more retaining elements that hold the syringe needle in the needle trap. For example, such retaining elements are designed as hook-shaped sections that are offset in relation to the recess so that the syringe needle can be deliberately pressed into the needle trap after use, bending the retaining elements away until the syringe needle is clipped in behind the retaining elements and secured in the recess of the needle trap provided for this purpose. Alternatively or additionally, the needle trap has one or more rigid retaining elements, which can contribute to the formation of a safe and reliable needle catcher or securing of the needle.

According to a preferred further embodiment of the labeling arrangement, the first and second label flaps and the label are formed in one piece from a common material web. In this way, a particularly simple and clear structure of the labeling arrangement can be realized with a small number of components. The common material web is provided, for example, as a film web from which the two label flaps and the label are punched out in a predetermined shape. In particular, the label can comprise an inscribable and/or printable area as a labeling medium or have an inscription and/or printing which, for example, comprises information about the syringe or its contents for which the labeling arrangement is intended.

The label, the first label flap and/or the second label flap can be single-layered or multi-layered. For example, the first label flap in particular has a two-layer structure in order to provide a reliable hold for the needle trap and to establish a more secure and stable joint for the needle trap. The second label flap can have one, two or more label layers and can include, for example, an adhesive or pressure-dependent structure, such as a void effect. When the syringe is opened, more adhesive sections can remain on the needle protection cap, while less adhesive sections remain on the syringe or the second label flap. Such a void effect can be used in particular to expose or make recognizable lettering that clearly indicates that the labeled syringe has already been opened for the first time. The described severability of the second label flap can therefore also include a severing or separation of sections or label layers of the second label flap, as is the case, for example, with a void effect, in which a label structure is detached or peeled off from another label structure and a predetermined pattern or lettering becomes visible.

The label, the first label flap and/or the second label flap preferably have a respective adhesive surface by means of which they can each be easily and reliably adhesively attached to the element provided for them consisting of the syringe body, needle trap and needle protection cap. The adhesive surfaces can be designed differently, they can be separated from each other and have an adhesive-free section in between or they can have a continuous adhesive surface on a common underside. Preferably, the adhesive surfaces are provided with adhesives that are pre-selected with regard to their use. For example, the adhesive surface of the label and the adhesive surface of the second label flap can have different adhesives, which are predetermined in accordance with the syringe body and the needle protection cap to which they are to be attached. Suitable adhesives for the label, the first label flap and/or for the second label flap can be pressure-sensitive adhesives and/or also heat-activated adhesives. Furthermore, hot-melt adhesives, construction adhesives and/or adhesives that can be cured by high-energy radiation can be useful.

According to a further preferred embodiment of the labeling arrangement, the second label flap has a predetermined severing area which is formed in coordination with a geometry of the syringe. The severing area comprises a severing element which is designed, for example, as a perforation and/or a punching or comprises such a perforation. The severing element is configured in particular in such a way that it indicates a successful opening of the syringe in a particularly recognizable manner. By means of the perforation or punching, a conspicuous tearing or tearing through of the second label flap can be provided in a targeted manner, so that a first removal of the needle protection cap is immediately apparent. For example, the perforation or punching is zig-zag shaped so that the separated sections of the second label flap are immediately recognizable.

Alternatively or additionally, the second label flap can have a predetermined colored area in the severing area, which is severed and/or exposed when the syringe is opened and the second label flap is severed. Preferably, the colored area comprises a particularly conspicuous signal color, so that after the needle protection cap is removed from the syringe needle, for example, a colored section of the second label flap remains on the syringe body and another colored section remains on the needle protection cap. Alternatively or additionally, such a colored section can be exposed. For example, the second label flap is multi-layered and has a neutral upper layer and a lower layer with a colored area underneath. When the syringe is opened, the upper layer is separated from the lower layer, exposing the colored area. The layer with the colored surface can be provided in the section of the second label flap that remains with the label and the syringe body or with the needle protection cap. Furthermore, the label and/or the first label flap can also be single-layered or multi-layered.

Alternatively or in addition to the colored section, the second label flap may also include a section that provides a void effect. Accordingly, a varnish area can be designed and integrated into the second label flap in such a way that it splits open at predefined points when it is peeled off, thereby revealing the lettering “void” or “open” or a symbol, for example, which can indicate an opening or attempted tampering.

The severing area forms a section of the second label flap in which irreversible destruction of the label flap is specifically initiated when the syringe is opened. The irreversible destruction can be achieved by suitable perforations and/or punching, which, for example, fan out particularly conspicuously after tearing or expose a conspicuous coloration of the second label flap. However, a relatively normal punching or perforation without special tearing structures and/or without color can also achieve a clearly recognizable proof of opening.

The severing area is preferably designed in accordance with the syringe so that, in relation to an applied state in which the labeling arrangement is operationally attached to the syringe, the severing area is arranged in a transition area between the syringe body and the needle protection cap. The severing area is thus located in the area of a syringe neck or a syringe shoulder and enables simple and reliable severing in this transition area. Preferably, the severing area in this transition area or in the area in which the severing element is formed has an adhesive-neutral or adhesive-free surface. This can contribute to a simple and reliable severing of the second label section.

According to a further preferred embodiment, the labeling arrangement comprises a carrier element which is coupled to the label, to the first label flap and to the second label flap. The carrier element has a straight contour on one edge and a curved contour with a recess on an opposite edge. The curved contour and the recess are predetermined in coordination with the first label flap and/or the needle trap, so that the first label flap is arranged in the area of the recess and the carrier element does not cover the first label flap or only covers it in sections on one side. Preferably, the curved contour and the recess are also predetermined in coordination with the second label flap, so that the second label flap is arranged adjacent to the recess and the carrier element covers the second label flap on one side.

The carrier element is configured, for example, as a silicone paper or siliconized film element and covers an underside of the label, the second label flap and possibly also a section of the first label flap. The edge of the carrier element with the described contours is formed in a predetermined manner, in particular by die-cutting from a provided starting material web. The specifically introduced recess forms a free area below the first label flap and the needle trap attached to it, so that particularly easy and convenient handling of the labeling arrangement can be achieved, especially when applying to a syringe.

The labeling arrangement can also be designed with respect to a longitudinal axis in such a way that the first and second label flaps are arranged in strips on a common side of the label at a predetermined distance from each other. The distance between the label flaps is predetermined, for example, for an intended insert on a syringe body, so that the distance corresponds to half the circumference of the syringe or the syringe body. The circumference is measured along the circumferential surface of the syringe radially around a longitudinal axis of the syringe, which essentially corresponds to the longitudinal axis of the labeling arrangement in relation to an operationally applied labeling arrangement. Accordingly, with respect to an applied state, the first label flap with the needle trap and the second label flap would be opposite each other with respect to the longitudinal axis and would be spaced as far apart as possible. Alternatively, a different distance can be formed so that the two label flaps are closer to each other and form separate sections.

Preferably, the second label flap has a length that is predetermined in accordance with the geometry of the needle protection cap. For example, the second label flap is L-shaped or T-shaped in relation to the longitudinal axis of the labeling arrangement. Furthermore, the first label flap and the needle trap can be designed to match each other in terms of their length and/or width. In addition, the label is also designed in a predetermined manner with regard to an intended use on the syringe barrel. For example, the label has a width transverse to the longitudinal axis that corresponds to a circumference of the syringe barrel. The label can therefore be a wrap-around label. Alternatively, the label can also be wider and form a wrap-around label and thus completely cover the circumferential surface of the syringe barrel radially and also lie on itself. Alternatively, the label can also have a smaller width and only cover a section of the outer surface of the syringe barrel.

The labeling arrangement is designed in particular for commercially available syringes in the medical-pharmaceutical sector. For example, the labeling arrangement is intended for a syringe with a volume of 1 ml, which has a commercially available syringe body with a diameter of 8.15 mm and a length of 54 mm. Accordingly, the label can be designed to fit such a lateral surface and cover a section of the lateral surface, enclose the lateral surface or be designed around it and have a width that is greater than the circumference of approximately 25.6 mm. Alternatively, the labeling arrangement can be provided for a syringe with a volume of 0.5 ml, which usually has a syringe body with a diameter of 6.85 mm and a length of 47.5 mm. Alternatively, the labeling arrangement can be provided for a syringe with a volume of 1, 1.5 ml or 2.25 ml, which, for example, has a syringe body with a diameter of 10.85 mm and a length of 36 mm, 43 mm or 54 mm.

In particular, a length and a width of the second label flap are also designed to match a geometry of an associated needle protection cap of the syringes described above. For example, the second label flap has a length of at least 5 mm in order to be able to form a reliable and secure hold on the needle protection cap. The length refers to a direction along the longitudinal axis towards the tip of the syringe needle. The second label flap is therefore a specifically extended section on the label, which cannot be easily and non-destructively lifted off when attached to the needle protection cap.

According to a further aspect, a syringe system comprises a syringe having a syringe body, a syringe needle and a needle protection cap surrounding the syringe needle. The syringe system further comprises an embodiment of the previously described labeling arrangement attached to a syringe, such that the label is arranged on the syringe body and the second label flap is arranged on the needle protection cap.

In that the syringe system comprises an embodiment of the described labeling arrangement, described properties and features of the labeling arrangement are also disclosed for the syringe system and vice versa.

According to a further aspect, a method for producing a labeling arrangement for a syringe, which has a syringe body, a syringe needle and a needle protection cap surrounding the syringe needle, comprises providing a label, a first label flap, a second label flap separate from the first label flap and a needle trap. The label is configured to be attached to the syringe body of the syringe. The needle trap is designed to receive and secure the syringe needle of the syringe after use.

The second label flap has a predetermined severable design and is adapted to be attached to the needle protection cap of the syringe and to form an opening detection element on the syringe. The method further comprises coupling the needle trap to the first label flap. Provided that the label, the first label flap and/or the second label flap are not provided in one piece, as a common component, the method may further comprise coupling the first and second label flaps to the label. In relation to an applied state in which the labeling arrangement is operationally attached to the syringe, when the syringe is opened by removing the needle protection cap, the second label flap can be severed and a section of the second label flap together with the needle protection cap can be removed from the label and the syringe body.

Using the method described, reliable protection with integrated proof of first opening for a syringe can be produced in a simple and cost-effective manner. In particular, the method described realizes a manufacturing method for making an embodiment of the labeling arrangement described above. Therefore, described properties and features of the labeling arrangement are also disclosed for the method and vice versa.

According to a preferred further embodiment of the method, the first and second label flap and the label are provided by forming them in one piece from a common web of material.

Furthermore, the method may comprise providing a carrier element which has a rectilinear contour on one edge and a curved contour with a recess on an opposite edge. The curved contour and the recess are predetermined in coordination with the first label flap and/or the needle trap. Accordingly, the method further comprises coupling the carrier element to the label, to the first label flap and to the second label flap, so that the first label flap is arranged in the area of the recess and the carrier element does not cover the first label flap or only covers it in sections on one side. Furthermore, the method can be further developed in such a way that the curved contour and the recess are also predetermined in coordination with the second label flap, so that after coupling the carrier element, the second label flap is arranged adjacent to the recess and the carrier element covers the second label flap on one side.

Alternatively, the carrier element and the label can also be provided and punched together so that coupling does not have to take place or has already taken place. In addition, the sequence of the specified process steps of the respective manufacturing method does not necessarily have to be carried out according to the sequence described in each case, but can also vary if possible. For example, the carrier element and an unpunched label web are joined together, and only then is the label with the two label flaps punched and formed from a top side, and a contour of the carrier element is produced from below.

According to a further preferred embodiment of the method, providing the label, the first and second label flap and/or the carrier element comprises providing a respective starting material web and die-cutting the starting material web, so that the label, the first and second label flap and/or the carrier element are each formed with a predetermined shape from the associated starting material web.

In particular, the method described can be configured to produce a plurality of the described labeling arrangements, for example by coupling and processing a continuous web of starting material for the carrier element and a continuous web of starting material for the label and the label flaps.

By means of the described design options for the labeling arrangement and for the manufacturing method, protection for syringes against external influences and tampering before use and also protection of a user against needlestick injury after use of the syringe can be provided in a common device in a cost-effective and space-saving manner. Therefore, no separate components are required, which have to be processed separately from each other and brought into contact with the syringe by the user. In this context, a user can be, for example, a packer or a pharmacist who attaches the labeling arrangement to a syringe in a predetermined manner. The user can also represent a doctor or a nurse who uses the labeled syringe. The labeling arrangement can be used to provide clear and reliable puncture and opening protection for syringes with a particularly small number of components. This means that separate syringe packaging can also be dispensed with, which, particularly in combination with separate needle stick protection, can result in a considerable amount of packaging material and also a considerable amount of space required for medication before it is administered.

The labeling arrangement combines a label with a needle trap with a first-opening seal, for example in the form of an additional, adhesive label flap, which is attached to the needle protection cap on the syringe by an adhesive during labeling. When the needle protection cap is removed, the adhesive tab is destroyed at least at one point according to its predetermined severability in such a way that this is later visible to a user and indicates that an opening has already taken place.

In addition, the specially designed carrier element in particular enables material-and cost-saving production of the labeling arrangement and simple, smooth dispensing of the labeling arrangement when labeling a syringe. The carrier element realizes a liner for the label and the label flaps. It can be particularly advantageous to specially design the second label flap in terms of its length and width and its shape in order to achieve particularly good adhesion to the needle protection cap or to achieve a particularly conspicuous representation of a first opening. The liner is preferably designed to match the respective shape of the label flaps and has a curved edge in order to expose the area of the labeling arrangement which is assigned to the first label flap and carries the needle trap and at the same time to keep the second label flap covered with the liner for the first opening indication.

In addition, the labeling arrangement can also have a carrier element with straight edges on both sides and/or a non-adhesive second label flap. An adhesive for connecting the second label flap to the needle protection cap can then, for example, only be applied either to the second label flap and/or to the needle protection cap during a labeling process. However, according to such an embodiment, the labeling arrangement also has a separate integral label section in addition to the label section for the needle trap, which is designed to be attached to the needle protection cap and to act as an opening proof. The label, the first label flap and the second label flap are realized in particular as film elements or film sections preferably of a common film web.

In the following, embodiments of the invention are explained with reference to schematic drawings. The figures shows:

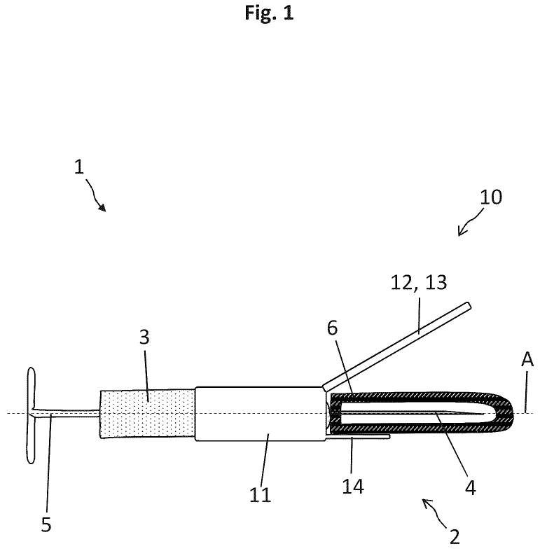

FIG. 1 an embodiment example of a syringe with a labeling arrangement applied thereto,

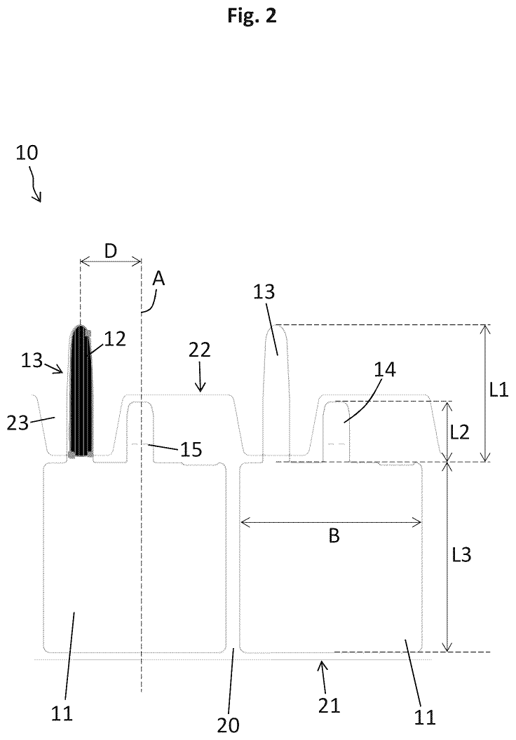

FIGS. 2-4 embodiments of the labeling arrangement for attaching to a syringe, and

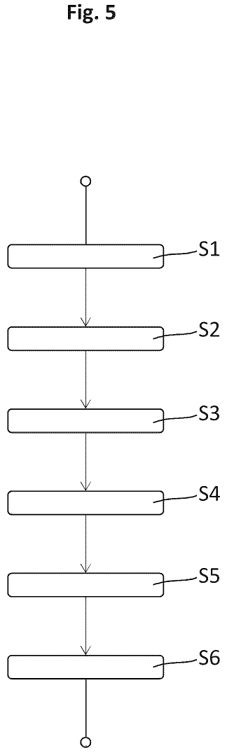

FIG. 5 a schematic flowchart for a method of manufacturing a labeling arrangement for a syringe.

Elements or features of the same construction or function are marked with the same reference signs across the figures. For reasons of clarity, not all of the elements or features shown in all of the figures are marked with the corresponding reference signs, possibly.

FIG. 1 shows a schematic side view of a syringe system with a syringe 1 and a labeling arrangement 10. The syringe 1 has a syringe body 3, a syringe needle 4 and a needle protection cap 6, which surrounds the syringe needle 4. By means of a syringe plunger 5, a content can be expelled from the syringe body 3 through the syringe needle 4 and injected into a patient. As will be explained below with reference to FIGS. 2 to 5, a reliable protection with an integrated first-opening detection for the syringe 1 is realized in a simple and cost-effective manner by means of the labeling arrangement 10.

The labeling arrangement 10 is designed to be attached to the syringe and comprises a label 11 which is attached to the syringe body 3, in particular by means of an adhesive. The labeling arrangement 10 also has a needle trap 12, which is designed to receive and secure the syringe needle 4 after use. The needle trap 12 is coupled, in particular by means of an adhesive, to a first label flap 13. The labeling arrangement 10 further comprises a second label flap 14 separate from the first label flap 13, which is designed to be separable or severable. The second label flap 14 is attached to the needle protection cap 6, in particular by means of an adhesive, and forms an opening detection element on the syringe 1. The first and second label flaps 13, 14 are connected to each other by means of the label 11, so that, in relation to an applied state in which the labeling arrangement 10 is attached to the syringe 1 in an operational state, when the syringe 1 is opened by removing the needle protection cap 6, the second label flap 14 is severed and a section of the second label flap 14 is removed together with the needle protection cap 6 from the label 11 and the syringe body 3.

The second label flap 14 can be formed as a single layer or have several label layers, so that it can also be possible to cut through by detaching one label layer from another. Alternatively or additionally, the second label layer 14 can have one or more printed layers, which are applied to a film element, for example, and can be cut through or separated from one another. Thus, at least one section of the second label flap 14 can be separated from a remaining part. This can be realized, for example, by the second label flap 14 comprising a void structure that provides a void effect.

FIG. 2 shows a schematic top view of an embodiment example of two labeling arrangements 10, which are arranged on a carrier element that realizes a liner 20. In the following description, mainly only one labeling arrangement 10 is described, the properties and features of which also apply to the other or other labeling arrangements 10. For reasons of clarity, not all elements are shown in all figures, so that, for example, in a final state of the labeling arrangement 10, a needle trap 12 is always coupled to an associated first label flap 13.

The second label flap 14 has a predetermined severing area with a severing element that is designed as a punching, slitting, creasing, notching and/or perforation 15. When the syringe 1 is opened and the needle protection cap 6 is pulled off, the second label flap 14 tears along the perforation 15 and is thus irreversibly destroyed. An opening can thus be indicated immediately and clearly and point to a syringe 1 that has been opened by mistake or without authorization.

The label 11, the first label flap 13 and the second label flap 14 are formed in one piece from a common material web. With respect to a longitudinal axis A of the labeling arrangement 10, the first label flap 13 and the second label flap 14 are formed in the shape of a strip on a common side of the label 11 with a predetermined distance D from each other. In an applied state, as illustrated in FIG. 1, the longitudinal axis A also corresponds to the longitudinal axis of the syringe 1. In particular, the distance D is designed to match the geometry of the syringe 1, so that the distance D corresponds, for example, to half the circumference of the syringe body 3. Thus, the first label flap 13 with the needle trap 12 of the second label flap 14 are opposite each other in relation to the longitudinal axis A.

A width B of the label 11 transverse to the longitudinal axis A is also preferably designed to match a circumference of the syringe 1 or the syringe barrel 3. For example, the label 11 can completely cover the circumference of the syringe barrel 3 radially and form a wrap-around label. If the width B is greater than the circumference of the syringe barrel 3, the label 11 forms a wrap-around label. However, the width B can also be smaller than the circumference of the syringe barrel 3, so that the label 11 covers a section of the outer surface of the syringe barrel 3.

The first label flap 13 has a length L1 which is designed in particular to match the geometry of the needle trap 12. The needle trap 12 is arranged on a side of the first label flap 13 that faces the syringe needle 4 with respect to an applied state. The needle trap 12 thus also faces the liner 20, which as a carrier element covers a respective adhesive on an underside of the label 11 and the second label flap 14. In particular, the needle trap 12 is designed to match the geometry of the syringe needle 4 so that it can securely hold and surround it.

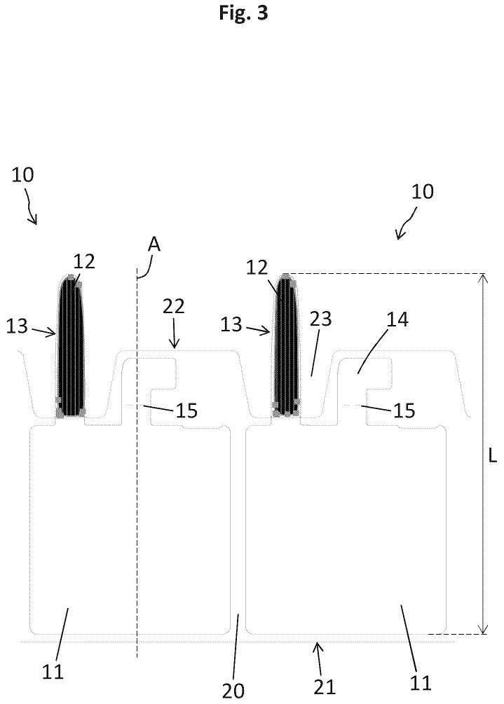

The second label flap 14 has a length L2, which is designed in particular to match a geometry of the needle protection cap 6. Furthermore, the second label flap 14 can be T-shaped or L-shaped (see FIG. 3) in order to provide a larger adhesive surface and enable better adhesion to the needle protection cap 6. A transverse section of a T-or L-shaped second label flap 14 can also be designed to match a circumference of the needle protection cap 6 and, like the label 11 on the syringe body 3, cover a section on the needle protection cap 6, surround it all around or even overlap it and come to rest with a section on itself.

The liner 20 is specially designed and has a straight contour 21 on one lateral edge and a curved contour 22 on an opposite lateral edge. The curved contour 22 comprises a recess 23, which is specifically designed in coordination with the first label flap 13 and the needle trap 12 coupled thereto. Thus, the first label flap 13 is arranged with the needle trap 12 in the area of the recess 23 and is not covered on one side or only in a lower section. Furthermore, the recess 23 is dimensioned in coordination with the second label flap 14 such that the second label flap 14 is arranged adjacent to the recess 23 and the carrier element 20 covers the second label flap 14 on one side (see FIGS. 2-4).

The label 11 has a length L3, which is designed in particular to match a length of the syringe barrel 3. The length L3 can correspond to the length of the syringe barrel 3 or be smaller or larger. The label 11 can be designed in such a way that it is relatively short and/or narrow in order to save material and costs, but large enough so that the intended information can be printed or applied.

Furthermore, the labeling arrangement 10 has an overall length L, which is essentially composed of the length L3 of the label 11 and the length L1 or L2 of the first or second label flap 13, 14. As an alternative to the illustrated embodiments, the second label flap 14 can also be longer than the first label flap 13 if required.

The length or width of the liner 20 is designed to match the geometry of the labeling arrangement 10. In this context, a width of the liner 20 denotes the extension that corresponds to the lengths L, L1, L2 and L3. A length of the liner 20 corresponds to an extension according to the width B, wherein the length of the liner 20 is usually formed continuously in the form of a web of material on which a plurality of labeling arrangements 10 are arranged or formed. A movement for processing such a web of material thus runs from left to right or vice versa according to the illustrated embodiment examples in FIGS. 2-4. The liner 20 and the labeling arrangement 10 are therefore geometrically matched to each other and to the intended use on the syringe 1.

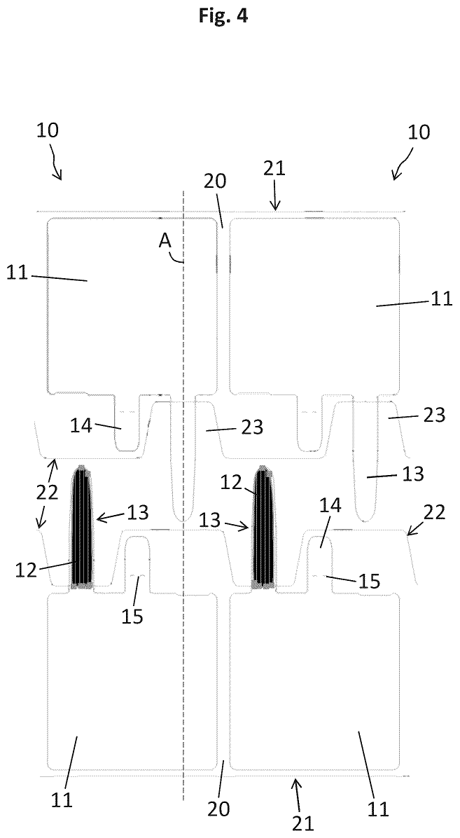

FIG. 4 shows a further embodiment example of labeling arrangements 10 that are designed opposite each other and offset with respect to the respective first label flaps 13 and needle traps 12. The width of the liner 20 is predetermined such that two labeling arrangements 10 located opposite each other can be accommodated on it. Such a width along the longitudinal axis A corresponds to slightly more than a composite length of L3+L2+L1+L3. In a central area, a stepped strip of the liner 20 is removed, for example by punching, so that an upper liner section and a lower liner section remain, each of which has a straight contour 21 and a curved contour 22 with equidistant recesses 23.

FIG. 5 schematically shows a flow chart for a method for manufacturing a design of the labeling arrangement 10 for the syringe 1. In a step S1, a starting material web, for example in the form of a film web, is provided. Furthermore, the needle trap 12 is provided, which is adapted to the syringe needle 4 to be secured. Furthermore, one or more further film webs can be provided in order to form a desired multilayer structure of the label 11, the first label flap 13 and/or the second label flap 14.

In a further step S2, the label 11, the first label flap 13 and the separate second label flap 14 are formed from the common film web by die-cutting.

In a step S3, the severing element is inserted into the second label flap 14. For example, the punching or perforation 15 is formed by punching or perforating, so that the second label flap can be predeterminedly destroyed.

In a further step S4, an adhesive is applied to an underside of the label 11 and the label flaps 13 and 14, whereby these can each have different adhesives with regard to their intended adhesion. Alternatively, the film web can already be provided with adhesive, so that the adhesive has already been applied.

In a further step S5, the needle trap 12 is coupled to the first label flap 13 by pressing the needle trap 12 against the adhesive underside of the first label flap 13.

In a further step S6, the liner 20 is provided and coupled to the label 11 and the label flaps 13 and 14, so that the first label flap 13 is arranged in the area of the recess 23 and the liner 20 does not cover the first label flap 13 or only covers it in sections on one side, and so that the second label flap 14 is arranged adjacent to the recess 23 and the liner 20 covers the second label flap 14 on one side. Providing the liner 20 may further comprise forming the liner 20 by means of punching, so that the described curved and straight contour 22, 21 is predetermined.

The described process sequence does not necessarily have to be carried out in the described order, but can also vary if possible. Furthermore, some of the steps described can also be carried out together in a common process. For example, the liner 20 can be provided together with the other components in step S1. The forming of the label 11, the first label flap 13, the second label flap 14 in the step S2 can be performed together with the insertion of the severing element into the second label flap 14 according to the step S3.

In particular, the method can be regarded as providing the label 11, the first and second label flaps 13, 14 and the liner 20 in the form of a respective starting material web so that a label roll with a plurality of labeling arrangements 10 are arranged on the curved liner 20.

The labeling arrangement 10 thus forms a combination of a needle protection label with a first opening indicator integrated therein. The second label flap 14 is preferably designed as an adhesive flap, which is connected to the needle protection cap 6 when the syringe 1 is unopened. When the needle protection cap 6 is opened and removed, the adhesive second label flap 14 is visibly changed, making the opening recognizable. To improve the dispensability of the labeling arrangement 10, the liner 20 is specially modified as a label carrier. The modified liner 20 has curved recesses 23 in the area of the needle traps 12, but covers the area of the second label flaps 14.

The labeling arrangement 10 forms a particularly space-saving, but also simple and cost-effective variant for combining pin-prick protection with first-opening protection, and also contributes to low material requirements and environmentally friendly production. The labeling arrangement 10 is suitable, among other things, when an increased space requirement, for example due to individual packaging, is disadvantageous. This applies, for example, to automatic medication dispensers in large pharmacies or hospitals, which dispense computer-controlled individual doses of medication that are packaged or labeled in a desirably slim and space-saving manner so that they can be dispensed smoothly.

The labeling arrangement 10 can be used just as advantageously in medical areas in which, for example, medication is already prepared and provided on a tray together with other medication and devices, whereby it should be ensured once again at the point of use that the syringe 1 with a needlestick protection that increases safety has not already been opened by mistake or without authorization.

### Reference Signs

1 syringe system
2 syringe
3 syringe body
4 syringe needle
5 syringe plunger
6 needle protection cap
10 labeling arrangement
11 label
12 needle trap
13 first label flap
14 second label flap/opening detection element
15 perforation/punching
20 carrier element/liner
21 straight contour
curved contour
recess
A longitudinal axis of the syringe system/labeling arrangement
B width of the label/labeling arrangement
D distance between the first and second label flap
L(i) length of a section of the labeling arrangement
L total length of the labeling arrangement

1. A labeling arrangement (10) for attachment to a syringe (1) comprising a syringe body (3), a syringe needle (4) and a needle protection cap (6) surrounding the syringe needle (4), the labelling arrangement (10) comprising:
a label (11) adapted to be attached to the syringe body (3) of the syringe (1),
a needle trap (12) adapted to receive and secure the syringe needle (4) of the syringe (1) after use,
a first label flap (13) coupled to the needle trap (12), and
a second label flap (14) separate from the first label flap (13), which is configured to be predeterminedly severable and is set up to be attached to the needle protection cap (6) of the syringe (1) and to form an opening detection element on the syringe (1), the first and second label flaps (13, 14) being connected to one another by means of the label (11), so that, with reference to an applied state in which the labeling arrangement (10) is attached to the syringe (1) in accordance with operation, when the syringe (1) is opened by removing the needle protection cap (6), the second label flap (14) can be severed and a section of the second label flap (14) together with the needle protection cap (6) can be removed from the label (11) and the syringe body (3).

2. The labeling arrangement (10) according to claim 1, wherein the first and second label flaps (13, 14) and the label (11) are formed in one piece from a common web of material.

3. The labeling arrangement (10) according to claim 1, wherein the label (11), the first label flap (13) and/or the second label flap (14) have an adhesive surface by means of which they can each be adhesively attached to the element provided for them comprising the syringe body (3), needle trap (12) and needle protection cap (6).

4. The labeling arrangement (10) according to claim 3, wherein the adhesive surface of the label (11) and the adhesive surface of the second label flap (14) have different adhesives which are designed in a predetermined manner in coordination with the syringe body (3) and the needle protection cap (6).

5. The labeling arrangement (10) according to claim 1, wherein the second label flap (14) has a predetermined severing area with a severing element (15) which is formed in coordination with a geometry of the syringe (1).

6. The labeling arrangement (10) according to claim 5, wherein the severing element (15) of the second label flap comprises a perforation and/or punching.

7. The labeling arrangement (10) according to claim 5, wherein the second label flap (14) has a predetermined colored area in the severing area, which is exposed when the syringe (1) is opened and the second label flap (14) is severed.

8. The labeling arrangement (10) according to claim 5, wherein the severing area is designed in a predetermined manner in coordination with the syringe (1), so that, with respect to an applied state in which the labeling arrangement (10) is attached to the syringe (1) during operation, the severing area is arranged in a transition region between the syringe body (3) and the needle protection cap (6).

9. The labeling arrangement (10) according to claim 5, wherein the severing area has an adhesive-neutral or adhesive-free surface in the region of the severing element (15).

10. The labeling arrangement (10) according to claim 1, comprising:
a carrier element (20) which is coupled to the label (11), to the first label flap (13) and to the second label flap (14), wherein the carrier element (20) has a straight contour (21) at one edge and a curved contour (22) at an opposite edge, and wherein the curved contour (22) comprises a recess (23), which is designed in a predetermined manner in coordination with the first label flap (13) and/or the needle trap (12), so that the first label flap (13) is arranged in the region of the recess (23) and the carrier element (20) does not cover the first label flap (13) or only covers it in sections on one side.

11. The labeling arrangement (10) according to claim 10, wherein the recess (23) is further configured in a predetermined manner in coordination with the second label flap (14), so that the second label flap (14) is arranged adjacent to the recess (23) and the carrier element (20) covers the second label flap (14) on one side.

12. The labeling arrangement (10) according to claim 1, wherein, with respect to a longitudinal axis (A) of the labeling arrangement (10), the first and the second label flap (13, 14) are formed in a strip-like manner on a common side of the label (11) at a predetermined distance from each other.

13. The labeling arrangement (10) according to claim 1, wherein, with respect to a longitudinal axis (A) of the labeling arrangement (10), the second label flap (14) has a length (L2) which is designed in a predetermined manner in coordination with a geometry of the needle protection cap (6).

14. The labeling arrangement (10) according to claim 1, wherein the second label flap (14) is L-shaped or T-shaped.

15. A syringe system, comprising:
a syringe (1) with a syringe body (3), a syringe needle (4) and a needle protection cap (6) surrounding the syringe needle (4), and
the labeling arrangement (10) according to one of the preceding claims claim 1, which is attached to a syringe (1) so that the label (11) is arranged on the syringe body (3) and the second label flap (14) is arranged on the needle protection cap (6).

16. A method for manufacturing a labeling arrangement (10) for a syringe (1), which has a syringe body (3), a syringe needle (4) and a needle protection cap (6) surrounding the syringe needle (4), the method comprising:
providing a label (11) adapted to be attached to the syringe body (3) of the syringe (1),
providing a needle trap (12) adapted to receive and secure the syringe needle (4) of the syringe (1) after use,
providing a first label flap (13),
providing a second label flap (14) which is separate from the first label flap (13), is designed to be predeterminedly severable and is adapted to be attached to the needle protection cap (6) of the syringe (1) and to form an opening detection element on the syringe (1), and
coupling the needle trap (12) to the first label flap (13), so that, with respect to an applied state in which the labeling arrangement (10) is attached to the syringe (1) in an operational manner, when the syringe (1) is opened by removing the needle protection cap (6), the second label flap (14) can be severed and a section of the second label flap (14) together with the needle protection cap (6) can be removed from the label (11) and the syringe body (3).

17. The method according to claim 16, wherein the first and second label flaps (13, 14) and the label (11) are provided by forming them in one piece from a common material web.

18. The method according to claim 16 or 17, comprising:
providing a carrier element (20) which has a rectilinear contour (21) at one edge and a curved contour (22) at an opposite edge, and wherein the curved contour (22) comprises a recess (23) which is formed in a predetermined manner in coordination with the first label flap (13) and/or the needle trap (12), and
coupling of the carrier element (20) to the label (11), to the first label flap (13) and to the second label flap (14), so that the first label flap (13) is arranged in the region of the recess (23) and the carrier element (20) does not cover the first label flap (13) or only covers it in sections on one side.

19. The method according to claim 18, wherein the recess (23) is further predeterminedly formed in coordination with the second label flap (14), so that after coupling the carrier element (20) the second label flap (14) is arranged adjacent to the recess (23) and the carrier element (20) covers the second label flap (14) on one side.

20. The method according to claim 16, wherein providing the label (11), the first and second label flaps (13, 14) and/or the carrier element (20) comprises:
providing a respective web of starting material, and
die-cutting the starting material web, so that the label (11), the first and second label flaps (13, 14) and/or the carrier element (20) are formed with a respective predetermined shape from the associated starting material web.
